(19) United States
(12) Patent Application Publication
(10) Pub. No.: US 2008/0090236 A1
(43) Pub. Date: Apr. 17, 2008

(54) Methods and systems for identifying tumor progression in comparative genomic hybridization data

(76) Inventors: Zohar H. Yakhini, Ramat Hasharon (IL); Doron Lipson, Rehovot (IL)

Correspondence Address: AGILENT TECHNOLOGIES INC., INTELLECTUAL PROPERTY ADMINISTRATION,LEGAL DEPT., MS BLDG. E P.O. BOX 7599, LOVELAND, CO, 80537, US

(21) Appl. No.: 11/580,802

(22) Filed: Oct. 13, 2006

(51) Int. Cl. C12Q 1/68; G06F 19/00

(52) U.S. Cl. 435/6; 702/20

(57) ABSTRACT

Methods for identification of statistically significant combinatorial patterns in CGH data that are indicative of the progression of chromosomal aberrations in tumors. The methods comprise acquiring comparative genomic hybridization data, identifying a long order preserving subset within the comparative genomic hybridization data, and identifying a chromosomal aberration associated with the long order preserving subset.

### FIELD OF THE INVENTION

The invention relates to methods for the identification of statistically significant combinatorial patterns in array-based comparative genomic hybridization (aCGH) data that are indicative of the progression of chromosomal aberrations in tumors.

### BACKGROUND OF THE INVENTION

Many events during the cell cycle can cause genomic instability through the deletion, duplication or translocation of DNA regions and result in alterations in DNA copy sequence number. Cancer progression often involves such changes in DNA copy number via over-expression of oncogenes and inactivation of tumor suppressor genes. Comparative genomic hybridization (CGH) is an important experimental technique that allows genome-wide analysis of DNA sequence copy number. The use of arrays for comparative genomic hybridization allows simultaneous evaluation of copy numbers at multiple positions across an entire genome, and provides a tool for clinical evaluation of cancer progression.

In a typical array CGH experiment, DNA from test cells is compared directly to DNA from normal cells. A glass slide or other array substrate is spotted with small DNA fragments from mapped genomic targets (i.e., DNA fragments of known identity and genomic position). A DNA test sample of interest and a DNA reference sample are each differentially labeled, and the combined test and reference samples are applied to the microarray. Intensity measurements for the genomic target sequences are then made to determine variations in copy number. Since the reference sample is generally diploid across the genome, target sequences with test intensities greater than the reference intensities indicate a gain in copy number, while lower intensities in the test sample indicate a loss in copy number. The gains and/or losses in copy numbers may indicate chromosomal alteration events associated with cancer.

There is accordingly a need for methods of evaluating comparative genomic hybridization data, and particularly array based comparative genomic hybridization data, for detection of chromosomal aberrations associated with cancer. The present invention satisfies this need, as well as others.

### RELEVANT LITERATURE

Relevant literature includes: Ben-Dor et al., RECOMB, 49-57, 2002; Pollack et al., Nature Genetics, 23(1):41-6, 1999; Saito et al., Molecular and Cellular Biology, 9(6):2445-2452, 1989; Sampas et. al., ATL DN 10020708, 2002; Yakhini et al., ATL DN 1004; 2004; U.S. Pat. No. 6,465,182; U.S. Pat. No. 6,335,167; U.S. Pat. No. 6,251,601; U.S. Pat. No. 6,210,878; U.S. Pat. No. 6,197,501; U.S. Pat. No. 6,159,685; U.S. Pat. No. 5,965,362; U.S. Pat. No. 5,830,645; U.S. Pat. No. 5,665,549; U.S. Pat. No. 5,447,841; U.S. Pat. No. 5,348,855; US2002/0006622; WO 99/23256; US20050234650; Pollack et al., Proc. Natl. Acad. Sci. (2002) 99: 12963-12968; Wilhelm et al., Cancer Res. (2002) 62: 957-960; Pinkel et al., Nat. Genet. (1998) 20: 207-211; Cai et al., Nat. Biotech. (2002) 20: 393-396; Snijders et al., Nat. Genet. (2001) 29:263-264; Hodgson et al., Nat. Genet. (2001) 29:459-464; Trask, Nat. Rev. Genet. (2002) 3: 769-778; Rabinovitch et al., Cancer Res. (1999) 59:5148-5153; Lee et al., Human Genet. (1997) 100:291:304; and Jong et al., Bioinformatics Advanced Access, Oxford University Press, Jul. 16, 2004; A. Ben-Dor, et al., Discovering local structure in gene expression data: The order-preserving submatrix problem. Journal of Computational Biology, 10(3-4):373-384, 2003; D. Aldous and P. Diaconis, Longest Increasing Subsequences: From Patience Sorting to the Baik-Dieft-Johansson Theorem, David Aldous, Bull. Amer. Math. Soc. 36, 413-32.

### SUMMARY OF THE INVENTION

The invention provides methods and systems for the identification of statistically significant combinatorial patterns in CGH data that are indicative of the progression of chromosomal aberrations in tumors. The methods are based on identifying long order preserving subsets (LOPS) in sets of vectors associated with CGH data. In general terms, the subject methods comprise acquiring comparative genomic hybridization data, identifying a long order preserving subset within the comparative genomic hybridization data, and identifying a chromosomal aberration associated with the long order preserving subset.

In certain embodiments of the invention the comparative genomic hybridization data may be comparative genomic hybridization array data.

In certain embodiments of the invention the methods may further comprise determining a nucleic acid copy number matrix from the comparative genomic hybridization array data, and identifying the long order preserving subset from nucleic acid copy number data vectors of the nucleic acid copy number matrix.

In certain embodiments of the invention the methods may further comprise selecting a continuous genomic segment and samples representing a subset of the nucleic acid copy number matrix, and identifying the long order preserving subset from nucleic copy number data vectors associated with the selected genomic segment and samples.

In certain embodiments of the invention the methods may further comprise identifying a genomic continuous order preserving submatrix associated with the selected genomic segment and samples.

Another aspect of the invention provides methods for tumor progression analysis in comparative genomic hybridization array data, the method comprising acquiring comparative genomic hybridization array data, defining a DNA copy number matrix C=G×S for the comparative genomic hybridization array data, wherein the matrix C comprises measured DNA copy number vectors for n chromosomal loci G over m samples S, identifying a long order preserving subset within the measured DNA copy number vectors, and identifying a chromosomal aberration associated with the long order preserving subset.

In certain embodiments of the invention the methods may further comprise selecting a continuous genomic segment G′ that is a subset of chromosomal locsi G, selecting a sample set sⱼ that is an element of samples S, such that continuous genomic segment G′ and sample set sⱼ define a submatrix of DNA copy number matrix C, and identifying the long order preserving subset from the DNA copy number vectors of the submatrix defined by the continuous genomic segment G′ and sample set sⱼ.

In certain embodiments of the invention the methods may further comprise defining a genomic continuous order preserving submatrix G′×L of DNA copy number matrix C, wherein L is the long order preserving subset.

In certain embodiments of the invention the methods may further comprise determining the penetrance of the chromosomal aberration within the subset of samples L.

Another aspect of the invention provides methods for tumor progression analysis in comparative genomic hybridization array data, the method comprising acquiring comparative genomic hybridization array data, defining a DNA copy number matrix C=G×S for the comparative genomic hybridization array data, wherein the matrix C comprises measured DNA copy number vectors for n chromosomal loci G over m samples S, selecting a continuous genomic segment G′=(gᵢ, . . . gᵢ₊ₖ) that is a subset of chromosomal locsi G, wherein gᵢ . . . gᵢ₊ₖ represent individual chromosomal loci, selecting a sample set sⱼ that is an element of samples S, wherein vector vⱼ for a DNA copy number measurement in continuous genomic segment G′ for the sample sⱼ is represented by vᵢ=[C(i,j), . . . C(i+k, j)], and identifying a long order preserving subset from a set of vectors v₁, . . . vₘ for the genomic segment G′.

In certain embodiments of the invention the methods may further comprise defining a genomic continuous order preserving submatrix G′×L wherein L is the long order preserving subset of samples S and G′ is the selected continuous genomic segment.

The invention also provides a tumor progression analysis system for comparative genomic hybridization data, the system comprising means for inputting comparative genomic hybridization data, means identifying a long order preserving subset within the comparative genomic hybridization data, and means for identifying a chromosomal aberration associated with the long order preserving subset. The comparative genomic hybridization data may comprise comparative genomic hybridization array data.

In certain embodiments the system may further comprise means for determining a nucleic acid copy number matrix from the comparative genomic hybridization array data, and means for identifying the long order preserving subset from nucleic acid copy number data vectors of the nucleic acid copy number matrix.

In certain embodiments the system may further comprise means for selecting a continuous genomic segment and samples representing a subset of the nucleic acid copy number matrix, and means for identifying the long order preserving subset from nucleic copy number data vectors associated with the selected genomic segment and samples.

In certain embodiments the system may further comprise means for identifying a genomic continuous order preserving submatrix associated with the selected genomic segment and samples.

The invention also provides a comparative genomic hybridization array data analysis system, and corresponding programming in a computer readable medium, comprising: means for measuring comparative genomic hybridization data; means for identifying a long order preserving subset within the comparative genomic hybridization data; and means for identifying a chromosomal aberration associated with the long order preserving subset.

The analysis system of the invention may also comprise means for determining a nucleic acid copy number matrix from the comparative genomic hybridization data, and means for identifying the long order preserving subset from the nucleic acid copy number data vectors of the nucleic acid copy number matrix.

The analysis system of the invention may further comprise means for selecting a genomic segment and samples representing a subset of the nucleic acid copy number matrix, and means for identifying the long order preserving subset from the nucleic acid copy number data vectors associated with the selected genomic segment and samples.

The analysis system of the invention may additionally comprise means for identifying a genomic continuous order preserving submatrix associated with the selected genomic segment and samples.

These and other advantages and features of the invention will become apparent to those persons skilled in the art upon reading the details more fully described below.

### BRIEF DESCRIPTION OF THE DRAWINGS

FIGS. 1A-D illustrate a LOPS within a two-dimensional distribution of data points.

FIGS. 2A-C illustrate a LOPS within a three-dimensional distribution of data points.

FIG. 3 is a graphical representation of the appearance of a LOPS in a DNA copy number data matrix.

FIG. 4 is a graphical comparison of the lengths of LOPS found in genomic continuous segments of breast tumor DNA copy number data.

FIG. 5 is a graphical representation of the appearance of a LOPS in a DNA copy number data matrix for an LOPS variant that allows the size of the vectors of the LOPS to monotonically decrease in magnitude.

FIG. 6 is a flow chart illustrating an embodiment of a method of the invention.

FIG. 7 is a block diagram illustrating an example of a computer system which may be used in implementing the present invention.

### DETAILED DESCRIPTION OF THE INVENTION

Before the present methods for tumor progression analysis of array CGH data are described, it is to be understood that this invention is not limited to particular genes or chromosomes described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are now described. All publications mentioned herein are incorporated herein by reference to disclose and describe the methods and/or materials in connection with which the publications are cited.

It should be noted that as used herein and in the appended claims, the singular forms “a”, “and”, and “the” include plural referents unless the context clearly dictates otherwise. Thus, for example, reference to “a polynucleotide” includes a plurality of such polynucleotides and reference to “the target fragment” includes reference to one or more target fragment and equivalents thereof known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed. All patents and publications mentioned herein are incorporated herein by reference in their entirety.

Definitions

The term “nucleic acid” and “polynucleotide” are used interchangeably herein to describe a polymer of any length, e.g., greater than about 10 bases, greater than about 100 bases, greater than about 500 bases, greater than 1000 bases, usually up to about 10,000 or more bases composed of nucleotides, e.g., deoxyribonucleotides or ribonucleotides, or compounds produced synthetically (e.g., PNA as described in U.S. Pat. No. 5,948,902 and the references cited therein) which can hybridize with naturally occurring nucleic acids in a sequence specific manner analogous to that of two naturally occurring nucleic acids, e.g., can participate in Watson-Crick base pairing interactions.

The terms “ribonucleic acid” and “RNA” as used herein mean a polymer composed of ribonucleotides.

The terms “deoxyribonucleic acid” and “DNA” as used herein mean a polymer composed of deoxyribonucleotides.

The term “oligonucleotide” as used herein denotes single stranded nucleotide multimers of from about 10 to 100 nucleotides and up to 200 nucleotides in length. Oligonucleotides are usually synthetic and, in many embodiments, are under 60 nucleotides in length.

The term “oligomer” is used herein to indicate a chemical entity that contains a plurality of monomers. As used herein, the terms “oligomer” and “polymer” are used interchangeably, as it is generally, although not necessarily, smaller “polymers” that are prepared using the functionalized substrates of the invention, particularly in conjunction with combinatorial chemistry techniques. Examples of oligomers and polymers include polydeoxyribonucleotides (DNA), polyribonucleotides (RNA), other nucleic acids that are C-glycosides of a purine or pyrimidine base, polypeptides (proteins), polysaccharides (starches, or polysugars), and other chemical entities that contain repeating units of like chemical structure.

The term “sample” as used herein relates to a material or mixture of materials, typically, although not necessarily, in fluid form, containing one or more components of interest.

The terms “nucleoside” and “nucleotide” are intended to include those moieties that contain not only the known purine and pyrimidine bases, but also other heterocyclic bases that have been modified. Such modifications include methylated purines or pyrimidines, acylated purines or pyrimidines, alkylated riboses or other heterocycles. In addition, the terms “nucleoside” and “nucleotide” include those moieties that contain not only conventional ribose and deoxyribose sugars, but other sugars as well. Modified nucleosides or nucleotides also include modifications on the sugar moiety, e.g., wherein one or more of the hydroxyl groups are replaced with halogen atoms or aliphatic groups, or are functionalized as ethers, amines, or the like.

The term “penetrance” refers to the degree to which the cells in a sample have been affected by the phenomenon being studied. Thus, for example, a tumor cell population in a sample having low penetrance is one in which not all of, or a relatively low percentage of, tumor cells have altered genomes.

The term “prevalence” refers to the degree to which all of the samples in a study have been affected by the phenomenon being studied. Thus, for example, a study showing low prevalence is one in which not all of, or a relatively low percentage of, samples in the study have altered genomes.

A “genomic-continuous set of loci” is a subset of the set of all measured loci, such that there is a chromosome such that all members of the subset are exactly the loci that reside in the chromosome and that have genomic positions between some given first and second genomic positions (i.e., between “genomic position a” and “genomic position b”).

A “continuous genomic segment” is a segment along a chromosome where a genomic-continuous set of loci are located.

The phrase “surface-bound polynucleotide” refers to a polynucleotide “probe” that is immobilized on a surface of a solid substrate, where the substrate can have a variety of configurations, e.g., a sheet, bead, or other structure. In certain embodiments, the collections of oligonucleotide probe elements employed herein are present on a surface of the same planar support, e.g., in the form of an array.

A “labeled population of nucleic acids” refers to mixture of nucleic acids that are detectably labeled, e.g., fluorescently labeled, such that the presence of the nucleic acids can be detected by assessing the presence of the label. A labeled population of nucleic acids is “made from” a chromosome sample, and the chromosome sample is usually employed as template for making the population of nucleic acids.

The term “array” encompasses the term “microarray” and refers to an ordered array presented for binding to nucleic acids and the like.

An “array,” includes any two-dimensional or substantially two-dimensional (as well as a three-dimensional) arrangement of spatially addressable regions bearing nucleic acid probes, particularly oligonucleotides or synthetic mimetics thereof, and the like. Where the arrays are arrays of nucleic acids, the nucleic acid probes may be adsorbed, physisorbed, chemisorbed, or covalently attached to the arrays at any point or points along the nucleic acid chain.

Any given substrate may carry one, two, four or more arrays disposed on a front surface of the substrate. Depending upon the use, any or all of the arrays may be the same or different from one another and each may contain multiple spots or features. A typical array may contain one or more, including more than two, more than ten, more than one hundred, more than one thousand, more ten thousand features, or even more than one hundred thousand features, in an area of less than 20 cm² or even less than 10 cm², e.g., less than about 5 cm², including less than about 1 cm², less than about 1 mm², e.g., 100 μm², or even smaller. For example, features may have widths (that is, diameter, for a round spot) in the range from a 10 μm to 1.0 cm. In other embodiments each feature may have a width in the range of 1.0 μm to 1.0 mm, usually 5.0 μm to 500 μm, and more usually 10 μm to 200 μm. Non-round features may have area ranges equivalent to that of circular features with the foregoing width (diameter) ranges. At least some, or all, of the features are of different compositions (for example, when any repeats of each feature composition are excluded the remaining features may account for at least 5%, 10%, 20%, 50%, 95%, 99% or 100% of the total number of features). Inter-feature areas will typically (but not essentially) be present which do not carry any nucleic acids (or other biopolymer or chemical moiety of a type of which the features are composed). Such inter-feature areas typically will be present where the arrays are formed by processes involving drop deposition of reagents but may not be present when, for example, photolithographic array fabrication processes are used. It will be appreciated though, that the inter-feature areas, when present, could be of various sizes and configurations.

Each array may cover an area of less than 200 cm², or even less than 50 cm², 5 cm², 1 cm², 0.5 cm², or 0.1 cm². In certain embodiments, the substrate carrying the one or more arrays will be shaped generally as a rectangular solid (although other shapes are possible), having a length of more than 4 mm and less than 150 mm, usually more than 4 mm and less than 80 mm, more usually less than 20 mm; a width of more than 4 mm and less than 150 mm, usually less than 80 mm and more usually less than 20 mm; and a thickness of more than 0.01 mm and less than 5.0 mm, usually more than 0.1 mm and less than 2 mm and more usually more than 0.2 and less than 1.5 mm, such as more than about 0.8 mm and less than about 1.2 mm. With arrays that are read by detecting fluorescence, the substrate may be of a material that emits low fluorescence upon illumination with the excitation light. Additionally in this situation, the substrate may be relatively transparent to reduce the absorption of the incident illuminating laser light and subsequent heating if the focused laser beam travels too slowly over a region. For example, the substrate may transmit at least 20%, or 50% (or even at least 70%, 90%, or 95%), of the illuminating light incident on the front as may be measured across the entire integrated spectrum of such illuminating light or alternatively at 532 nm or 633 mm.

Arrays can be fabricated using drop deposition from pulse-jets of either nucleic acid precursor units (such as monomers) in the case of in situ fabrication, or the previously obtained nucleic acid. Such methods are described in detail in, for example, the previously cited references including U.S. Pat. No. 6,242,266, U.S. Pat. No. 6,232,072, U.S. Pat. No. 6,180,351, U.S. Pat. No. 6,171,797, U.S. Pat. No. 6,323,043, U.S. patent application Ser. No. 09/302,898 filed Apr. 30, 1999 by Caren et al., and the references cited therein. These references are incorporated herein by reference. Other drop deposition methods can be used for fabrication, as previously described herein. Also, instead of drop deposition methods, photolithographic array fabrication methods may be used. Inter-feature areas need not be present particularly when the arrays are made by photolithographic methods as described in those patents.

An array is “addressable” when it has multiple regions of different probes (e.g., different oligonucleotide sequences) such that a region (i.e., a “feature” or “spot” of the array) at a particular predetermined location (i.e., an “address”) on the array will detect a particular target sequence. Array features are typically, but need not be, separated by intervening spaces. It should be noted that the terms “target” and “probe” are sometimes used differently in certain publications.

A “scan region” refers to a contiguous (preferably, rectangular) area in which the array spots, probes or features of interest are found or detected. Where fluorescent labels are employed, the scan region is that portion of the total area illuminated from which the resulting fluorescence is detected and recorded. Where other detection protocols are employed, the scan region is that portion of the total area queried from which resulting signal is detected and recorded. For the purposes of this invention and with respect to fluorescent detection embodiments, the scan region includes the entire area of the slide scanned in each pass of the lens, between the first feature of interest, and the last feature of interest, even if there exist intervening areas that lack features of interest.

An “array layout” refers to one or more characteristics of the features, such as feature positioning on the substrate, one or more feature dimensions, and an indication of a moiety at a given location. “Hybridizing” and “binding”, with respect to nucleic acids, are used interchangeably.

The term “stringent assay conditions” as used herein refers to conditions that are compatible to produce binding pairs of nucleic acids, e.g., probes and targets, of sufficient complementarity to provide for the desired level of specificity in the assay while being incompatible to the formation of binding pairs between binding members of insufficient complementarity to provide for the desired specificity. The term stringent assay conditions refers to the combination of hybridization and wash conditions.

A “stringent hybridization” and “stringent hybridization wash conditions” in the context of nucleic acid hybridization (e.g., as in array, Southern or Northern hybridizations) are sequence dependent, and are different under different environmental parameters. Stringent hybridization conditions that can be used to identify nucleic acids within the scope of the invention can include, e.g., hybridization in a buffer comprising 50% formamide, 5×SSC, and 1% SDS at 42° C., or hybridization in a buffer comprising 5×SSC and 1% SDS at 65° C., both with a wash of 0.2×SSC and 0.1% SDS at 65° C. Exemplary stringent hybridization conditions can also include a hybridization in a buffer of 40% formamide, 1 M NaCl, and 1% SDS at 37° C., and a wash in 1×SSC at 45° C. Alternatively, hybridization to filter-bound DNA in 0.5 M NaHPO₄, 7% sodium dodecyl sulfate (SDS), 1 nmM EDTA at 65° C., and washing in 0.1×SSC/0.1% SDS at 68° C. can be employed. Yet additional stringent hybridization conditions include hybridization at 60° C. or higher and 3×SSC (450 mM sodium chloride/45 mM sodium citrate) or incubation at 42° C. in a solution containing 30% formamide, 1M NaCl, 0.5% sodium sarcosine, 50 mM MES, pH 6.5. Those of ordinary skill will readily recognize that alternative but comparable hybridization and wash conditions can be utilized to provide conditions of similar stringency.

In certain embodiments, the stringency of the wash conditions determine whether a nucleic acid is specifically hybridized to a probe. Wash conditions used to identify nucleic acids may include, e.g.: a salt concentration of about 0.02 molar at pH 7 and a temperature of at least about 50° C. or about 55° C. to about 60° C.; or, a salt concentration of about 0.15 M NaCl at 72° C. for about 15 minutes; or, a salt concentration of about 0.2×SSC at a temperature of at least about 50° C. or about 55° C. to about 60° C. for about 15 to about 20 minutes; or, the hybridization complex is washed twice with a solution with a salt concentration of about 2×SSC containing 0.1% SDS at room temperature for 15 minutes and then washed twice by 0.1×SSC containing 0.1% SDS at 68° C. for 15 minutes; or, equivalent conditions. Stringent conditions for washing can also be, e.g., 0.2×SSC/0.1% SDS at 42° C. In instances wherein the nucleic acid molecules are deoxyoligonucleotides (“oligos”), stringent conditions can include washing in 6×SSC/0.05% sodium pyrophosphate at 37° C. (for 14-base oligos), 48° C. (for 17-base oligos), 55° C. (for 20-base oligos), and 60° C. (for 23-base oligos). See Sambrook, Ausubel, or Tijssen (cited below) for detailed descriptions of equivalent hybridization and wash conditions and for reagents and buffers, e.g., SSC buffers and equivalent reagents and conditions.

Stringent hybridization conditions may also include a “prehybridization” of aqueous phase nucleic acids with complexity-reducing nucleic acids to suppress repetitive sequences. For example, certain stringent hybridization conditions include, prior to any hybridization to surface-bound polynucleotides, hybridization with Cot-1 DNA, or the like.

Stringent assay conditions are hybridization conditions that are at least as stringent as the above representative conditions, where a given set of conditions are considered to be at least as stringent if substantially no additional binding complexes that lack sufficient complementarity to provide for the desired specificity are produced in the given set of conditions as compared to the above specific conditions, where by “substantially no more” is meant less than about 5-fold more, typically less than about 3-fold more. Other stringent hybridization conditions are known in the art and may also be employed, as appropriate.

“CGH” or “Comparative Genomic Hybridization” refers generally to techniques for identification of chromosomal alterations (such as in cancer cells, for example). Using CGH, ratios between tumor or test sample and normal or control sample enable the detection of chromosomal amplifications and deletions of regions that may include oncogenes and tumor suppressive genes, for example.

A “CGH array” or “aCGH array” refers to an array that can be used to compare DNA samples for relative differences in copy number. In general, an aCGH array can be used in any assay in which it is desirable to scan a genome with a sample of nucleic acids. For example, an aCGH array can be used in location analysis as described in U.S. Pat. No. 6,410,243, the entirety of which is incorporated herein. In certain aspects, a CGH array provides probes for screening or scanning a genome of an organism and comprises probes from a plurality of regions of the genome. In one aspect, the array comprises probe sequences for scanning an entire chromosome arm, wherein probes targets are separated by at least about 500 bp, at least about 1 kb, at least about 5 kb, at least about 10 kb, at least about 25 kb, at least about 50 kb, at least about 100 kb, at least about 250 kb, at least about 500 kb and at least about 1 Mb. In another aspect, the array comprises probe sequences for scanning an entire chromosome, a set of chromosomes, or the complete complement of chromosomes forming the organism's genome. By “resolution” is meant the spacing on the genome between sequences found in the probes on the array. In some embodiments (e.g., using a large number of probes of high complexity) all sequences in the genome can be present in the array. The spacing between different locations of the genome that are represented in the probes may also vary, and may be uniform, such that the spacing is substantially the same between sampled regions, or non-uniform, as desired. An assay performed at low resolution on one array, e.g., comprising probe targets separated by larger distances, may be repeated at higher resolution on another array, e.g., comprising probe targets separated by smaller distances.

In certain aspects, in constructing the arrays, both coding and non-coding genomic regions are included as probes, whereby “coding region” refers to a region comprising one or more exons that is transcribed into an mRNA product and from there translated into a protein product, while by non-coding region is meant any sequences outside of the exon regions, where such regions may include regulatory sequences, e.g., promoters, enhancers, untranslated but transcribed regions, introns, origins of replication, telomeres, etc. In certain embodiments, one can have at least some of the probes directed to non-coding regions and others directed to coding regions. In certain embodiments, one can have all of the probes directed to non-coding sequences. In certain embodiments, one can have all of the probes directed to coding sequences. In certain other aspects, individual probes comprise sequences that do not normally occur together, e.g., to detect gene rearrangements, for example.

In some embodiments, at least 5% of the polynucleotide probes on the solid support hybridize to regulatory regions of a nucleotide sample of interest while other embodiments may have at least 30% of the polynucleotide probes on the solid support hybridize to exonic regions of a nucleotide sample of interest. In yet other embodiments, at least 50% of the polynucleotide probes on the solid support hybridize to intergenic (e.g., non-coding) regions of a nucleotide sample of interest. In certain aspects, probes on the array represent random selection of genomic sequences (e.g., both coding and noncoding). However, in other aspects, particular regions of the genome are selected for representation on the array, e.g., such as CpG islands, genes belonging to particular pathways of interest or whose expression and/or copy number are associated with particular physiological responses of interest (e.g., disease, such a cancer, drug resistance, toxological responses and the like). In certain aspects, where particular genes are identified as being of interest, intergenic regions proximal to those genes are included on the array along with, optionally, all or portions of the coding sequence corresponding to the genes. In one aspect, at least about 100 bp, 500 bp, 1,000 bp, 5,000 bp, 10,000 kb or even 100,000 kb of genomic DNA upstream of a transcriptional start site is represented on the array in discrete or overlapping sequence probes. In certain aspects, at least one probe sequence comprises a motif sequence to which a protein of interest (e.g., such as a transcription factor) is known or suspected to bind.

In certain aspects, repetitive sequences are excluded as probes on the arrays. However, in another aspect, repetitive sequences are included.

The choice of nucleic acids to use as probes may be influenced by prior knowledge of the association of a particular chromosome or chromosomal region with certain disease conditions. International Application WO 93/18186 provides a list of exemplary chromosomal abnormalities and associated diseases, which are described in the scientific literature. Alternatively, whole genome screening to identify new regions subject to frequent changes in copy number can be performed using the methods of the present invention discussed further below.

In some embodiments, previously identified regions from a particular chromosomal region of interest are used as probes. In certain embodiments, the array can include probes which “tile” a particular region (e.g., which have been identified in a previous assay or from a genetic analysis of linkage), by which is meant that the probes correspond to a region of interest as well as genomic sequences found at defined intervals on either side, i.e., 5′ and 3′ of, the region of interest, where the intervals may or may not be uniform, and may be tailored with respect to the particular region of interest and the assay objective. In other words, the tiling density may be tailored based on the particular region of interest and the assay objective. Such “tiled” arrays and assays employing the same are useful in a number of applications, including applications where one identifies a region of interest at a first resolution, and then uses tiled array tailored to the initially identified region to further assay the region at a higher resolution, e.g., in an iterative protocol.

“Themed” arrays may be fabricated, for example, arrays including whose duplications or deletions are associated with specific types of cancer (e.g., breast cancer, prostate cancer and the like). The selection of such arrays may be based on patient information such as familial inheritance of particular genetic abnormalities. In certain aspects, an array for scanning an entire genome is first contacted with a sample and then a higher-resolution array is selected based on the results of such scanning.

Themed arrays also can be fabricated for use in gene expression assays, for example, to detect expression of genes involved in selected pathways of interest, or genes associated with particular diseases of interest.

In one embodiment, a plurality of probes on the array are selected to have a duplex Tₘ within a predetermined range. For example, in one aspect, at least about 50% of the probes have a duplex Tₘ within a temperature range of about 75° C. to about 85° C. In one embodiment, at least 80% of said polynucleotide probes have a duplex Tₘ within a temperature range of about 75° C. to about 85° C., within a range of about 77° C. to about 83° C., within a range of from about 78° C. to about 82° C. or within a range from about 79° C. to about 82° C. In one aspect, at least about 50% of probes on an array have range of Tₘ's of less than about 4° C., less then about 3° C., or even less than about 2° C., e.g., less than about 1.5° C., less than about 1.0° C. or about 0.5° C.

The probes on the microarray, in certain embodiments have a nucleotide length in the range of at least 30 nucleotides to 200 nucleotides, or in the range of at least about 30 to about 150 nucleotides. In other embodiments, at least about 50% of the polynucleotide probes on the solid support have the same nucleotide length, and that length may be about 60 nucleotides.

In certain aspects, longer polynucleotides may be used as probes. In addition to the oligonucleotide probes described above, cDNAs, or inserts from phage BACs (bacterial artificial chromosomes) or plasmid clones, can be arrayed. Probes may therefore also range from about 201-5000 bases in length, from about 5001-50,000 bases in length, or from about 50,001-200,000 bases in length, depending on the platform used. If other polynucleotide features are present on a subject array, they may be interspersed with, or in a separately-hybridizable part of the array from the subject oligonucleotides.

In still other aspects, probes on the array comprise at least coding sequences.

In one aspect, probes represent sequences from an organism such as Drosophila melanogaster, Caenorhabditis elegans, yeast, zebrafish, a mouse, a rat, a domestic animal, a companion animal, a primate, a human, etc. In certain aspects, probes representing sequences from different organisms are provided on a single substrate, e.g., on a plurality of different arrays.

A “CGH assay” using an aCGH array can be generally performed as follows. In one embodiment, a population of nucleic acids contacted with an aCGH array comprises at least two sets of nucleic acid populations, which can be derived from different sample sources. For example, in one aspect, a target population contacted with the array comprises a set of target molecules from a reference sample and from a test sample. In one aspect, the reference sample is from an organism having a known genotype and/or phenotype, while the test sample has an unknown genotype and/or phenotype or a genotype and/or phenotype that is known and is different from that of the reference sample. For example, in one aspect, the reference sample is from a healthy patient while the test sample is from a patient suspected of having cancer or known to have cancer.

In one embodiment, a target population being contacted to an array in a given assay comprises at least two sets of target populations that are differentially labeled (e.g., by spectrally distinguishable labels). In one aspect, control target molecules in a target population are also provided as two sets, e.g., a first set labeled with a first label and a second set labeled with a second label corresponding to first and second labels being used to label reference and test target molecules, respectively.

In one aspect, the control target molecules in a population are present at a level comparable to a haploid amount of a gene represented in the target population. In another aspect, the control target molecules are present at a level comparable to a diploid amount of a gene. In still another aspect, the control target molecules are present at a level that is different from a haploid or diploid amount of a gene represented in the target population. The relative proportions of complexes formed labeled with the first label vs. the second label can be used to evaluate relative copy numbers of targets found in the two samples.

In certain aspects, test and reference populations of nucleic acids may be applied separately to separate but identical arrays (e.g., having identical probe molecules) and the signals from each array can be compared to determine relative copy numbers of the nucleic acids in the test and reference populations.

Following receipt by a user, an array will typically be exposed to a sample and then read. Reading of an array may be accomplished by illuminating the array and reading the location and intensity of resulting fluorescence at multiple regions on each feature of the array. For example, a scanner that may be used for this purpose is the AGILENT MICROARRAY SCANNER manufactured by Agilent Technologies, Palo, Alto, Calif. or other similar scanner. Other suitable apparatus and methods are described in U.S. Pat. Nos. 6,518,556; 6,486,457; 6,406,849; 6,371,370; 6,355,921; 6,320,196; 6,251,685 and 6,222,664. Scanning typically produces a scanned image of the array which may be directly inputted to a feature extraction system for direct processing and/or saved in a computer storage device for subsequent processing. However, arrays may be read by any other methods or apparatus than the foregoing, other reading methods including other optical techniques or electrical techniques (where each feature is provided with an electrode to detect bonding at that feature in a manner disclosed in U.S. Pat. Nos. 6,251,685, 6,221,583 and elsewhere).

The term “mixture”, as used herein, refers to a combination of elements, that are interspersed and not in any particular order. A mixture is heterogeneous and not spatially separable into its different constituents. Examples of mixtures of elements include a number of different elements that are dissolved in the same aqueous solution, or a number of different elements attached to a solid support at random or in no particular order in which the different elements are not specially distinct. In other words, a mixture is not addressable. To be specific, an array of surface-bound polynucleotides, as is commonly known in the art and described below, is not a mixture of capture agents because the species of surface-bound polynucleotides are spatially distinct and the array is addressable.

“Isolated” or “purified” generally refers to isolation of a substance (compound, polynucleotide, protein, polypeptide, polypeptide, chromosome, etc.) such that the substance comprises the majority percent of the sample in which it resides. Typically in a sample a substantially purified component comprises 50%, preferably 80%-85%, more preferably 90-95% of the sample. Techniques for purifying polynucleotides, polypeptides and intact chromosomes of interest are well-known in the art and include, for example, ion-exchange chromatography, affinity chromatography, sorting, and sedimentation according to density.

The terms “assessing” and “evaluating” are used interchangeably to refer to any form of measurement, and include determining if an element is present or not. The terms “determining,” “measuring,” and “assessing,” and “assaying” are used interchangeably and include both quantitative and qualitative determinations. Assessing may be relative or absolute. “Assessing the presence of” includes determining the amount of something present, as well as determining whether it is present or absent.

If a surface-bound polynucleotide “corresponds to” a genomic region, the polynucleotide usually contains a sequence of nucleic acids that is unique to that region. Accordingly, a surface-bound polynucleotide that corresponds to a particular chromosome usually specifically hybridizes to a labeled nucleic acid made from that chromosome, relative to labeled nucleic acids made from other chromosomes. Array features, because they usually contain surface-bound polynucleotides, can also correspond to a chromosome.

### Methods

The methods of the invention are described in terms of use with data derived from arrays or microarrays. It should be understood, however, that the invention may be used with any data that carries genomic copy number data, including data derived from arrays, polymerase chain reaction (PCR) experiments, cell sorting, or other techniques. The invention is also described in terms of use with DNA-based arrays, and it is contemplated that the invention may be used with data generated from RNA-based arrays as well.

The invention is particularly useful in association with arrays capable of providing genomic copy number information. Arrays suitable for use in performing the subject methods may, for example, contain a plurality (i.e., at least about 100, at least about 500, at least about 1000, at least about 2000, at least about 5000, at least about 10,000, at least about 20,000, usually up to about 100,000 or more) of addressable features containing oligonucleotides that are linked to a usually planar solid support such as a glass or silicon substrate. Features on an array usually contain polynucleotides that hybridize to, i.e., bind to, genomic sequences from a cell. Such comparative genome hybridization arrays typically include a plurality of different oligonucleotides that are addressably arrayed. The array features may also contain other polynucleotides, such as cDNAs, or inserts from phage BACs (bacterial artificial chromosomes) or plasmid clones. While the CGH arrays usually contain features of oligonucleotides, they may also contain features of polynucleotides that are about 201-5000 bases in length, about 5001-50,000 bases in length, or about 50,001-200,000 bases in length, depending on the platform used. If other polynucleotide features are present on a subject array, they may be interspersed with, or in a separately-hybridizable part of the array from, the subject oligonucleotides.

The arrays used with the invention may be prepared by a variety of well-known techniques, including drop deposition from pulse jets or from fluid-filled tips, etc, or using photolithographic means. Polynucleotide precursor units (such as nucleotide monomers), in the case of in situ fabrication, or a previously synthesized polynucleotides (e.g., oligonucleotides, amplified cDNAs or isolated BAC, bacteriophage and plasmid clones, and the like) can be deposited on arrays. Common array fabrication techniques are described in U.S. Pat. No. 6,242,266, U.S. Pat. No. 6,232,072, U.S. Pat. No. 6,180,351, U.S. Pat. No. 6,171,797, and U.S. Pat. No. 6,323,043.

The methods of the invention utilize long order preserving subsets (LOPS) in sets of DNA copy number data points or features. Data features in microarray data sets have both identities and values. The value of a data feature is generally a measure of scanned intensities of light or radiation emitted from labeled target molecules bound to the feature. The identity may be based on two-coordinate indexes, a sequence number, or an alphanumeric label that uniquely identifies the feature within the data set. A data point may also, in some embodiments of the invention, be associated with a weight, where the weight expresses a measure of confidence, constancy, or some other parameter or characteristic.

An order-preserving sequence is a sequence of data points or features in which the values of the data points within the sequence uniformly increase within the sequence. When a sequence is defined as an ordered subset of points within a data set, then a longest-order-preserving subset or LOPS is the maximally sized subset of points selected from the data set that are ordered by signal strength or by some other associated value, parameter, or characteristic.

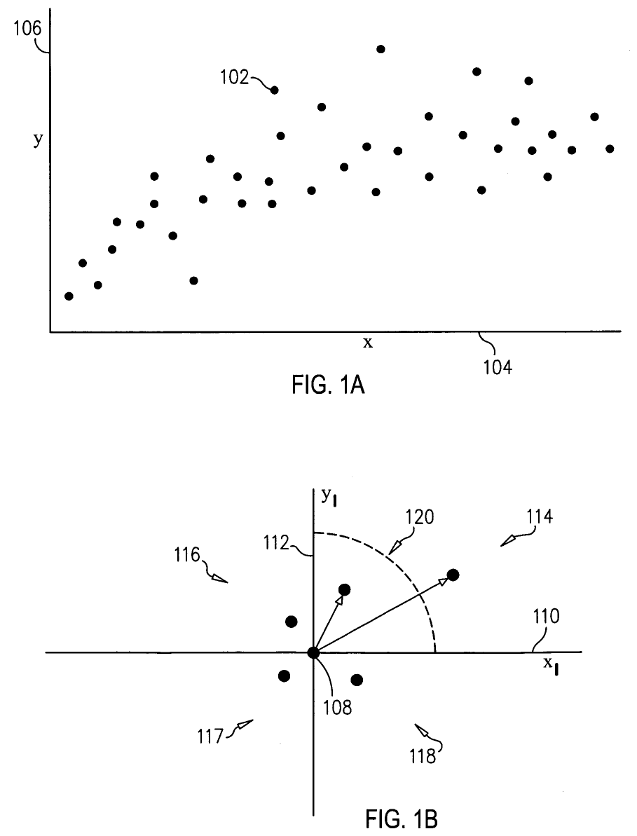

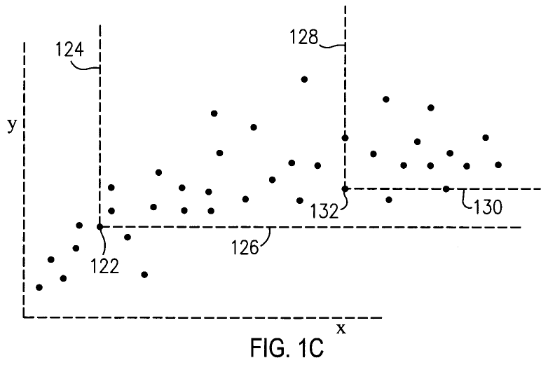

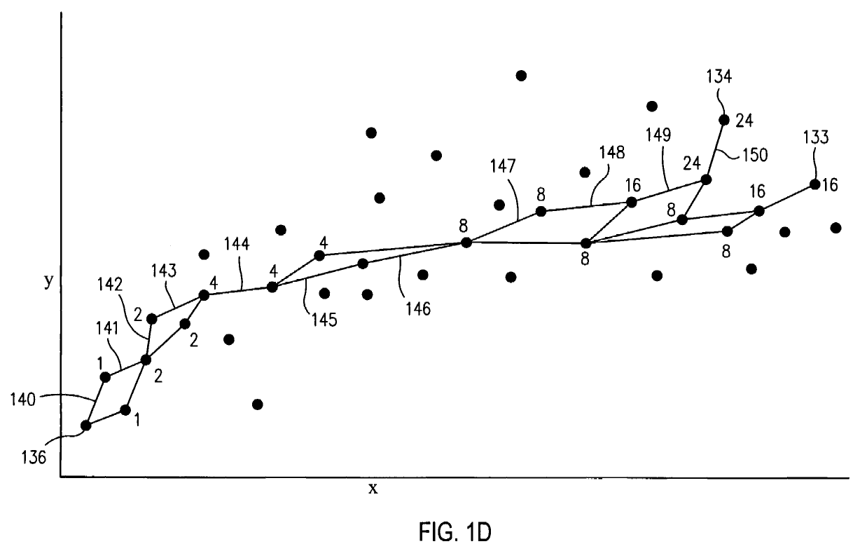

FIGS. 1A-D illustrate a two-dimensional LOPS. In FIG. 1A, a number of data points, such as data point 1002, are distributed in a two-dimensional space defined by an orthogonal coordinate system. The positive, horizontal axis 104 corresponds to a first coordinate x, and the vertical axis 106 in FIG. 1A is the positive axis for the coordinate y. Each data point, such as data point 102, has an identity as well as an x value and a y value represented by the position of the data point within the two-dimensional space. Commonly, the data points are associated with Cartesian coordinates (x, y) where x is the value of the data point with respect to the x axis, and y is the value of the data point with respect to the y axis. Such a two-dimensional distribution may arise from scanning a CGH microarray at two different frequencies, with the x values representing signal intensities scanned at one frequency, and the y values representing signal intensities scanned at another frequency.

To determine a LOPS for a two-dimensional distribution of data points, such as that shown in FIG. 1A, a means of establishing the order of data points must be first defined. FIG. 1B illustrates one technique for establishing the order of data points within a two-dimensional distribution, such as that shown in FIG. 1A. In FIG. 1B, a data point 108 is shown at the position (0,0) with respect to local, orthogonal axes xₗ 110 and yₗ 112. Any data point in the first quadrant of the two-dimensional space defined by orthogonal axes xₗ and yₗ is considered to be greater than, or having a higher order than, data point 108. Data points within the diagonally opposite quadrant 117 of the two-dimensional space are considered to be less than, or of smaller order than, data point 108. Data points in the neighboring quadrants 116 and 118 are not considered, as they are not directly comparable by a less-than/greater-than ordering, so the defined ordering is a partial ordering. In other words, an order-preserving sequence, or path, in a two-dimensional space defined by orthogonal coordinates has path edges that point in a 90-degree 120 range of directions between east, corresponding to the direction of the x-coordinate axis, and north, corresponding to the direction of the positive y-coordinate axis.

In FIG. 1C, the definition of order illustrated in FIG. 1B is applied to each data point within the data point distribution and selects a subset of data points that are greater than any particular data point. For example, all data points to the right of local y axis 124 and above local x axis 126 are considered to be greater than data point 122, and all data points to the right of local y axis 128 and above local x axis 130 are considered to be greater than data point 132. This definition allows for constructing or identifying LOPS within a two-dimensional distribution of data points. Since the signals in each dimension are compared within each dimension independently, only the ranking of the points is necessary to determine the LOPS. Thus, the ranks of the points in each dimension can be substituted for their real experimental values.

FIG. 1D illustrates construction of a number of longest order-preserving subsets within the distribution of data points shown in FIG. 1A. There may be many different longest order-preserving subsets that all have the same, maximal length, within a two-dimensional distribution of data points. Thus there may be many different LOPS within a two-dimensional data-point distribution. In FIG. 1B, the numerical label associated with certain data points, such as numerical label “24” associated with data point 134, indicates the number of different LOPS that begin with data point 1036 and end with the data point having the associated numerical label. Thus, there are 24 longest order-preserving sequences of length 12 (where length equals the number of nodes in a sequence) that begin with data point 136 and end with data point 134. There are 16 different LOPS that begin with data point 136 and end with data point 138. There are 40 different LOPS with maximal length of 12 shown within the two-dimensional data distribution of FIG. 1D. Any one of these 40 different LOPS in FIG. 1D can be constructed from edges 140-150. While all LOPS shown in FIG. 1D begin with the single data point 136, all LOPS constructed from a general data-point distribution need not begin with a particular point.

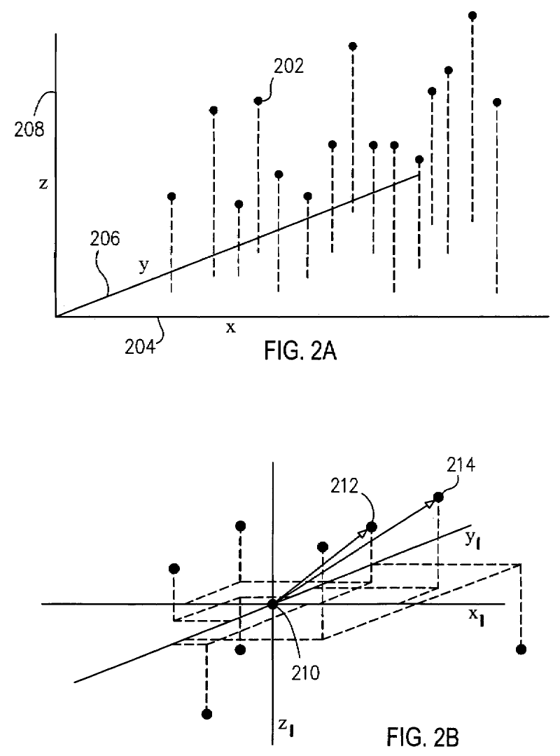

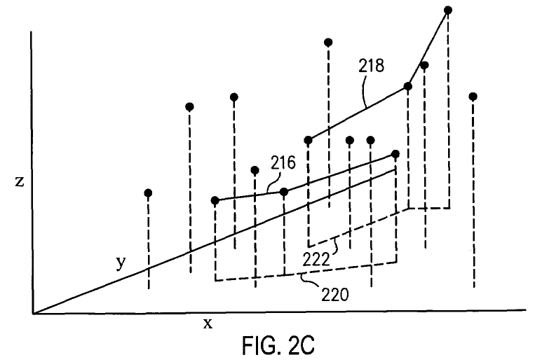

FIGS. 2A-C illustrate a LOPS within a three-dimensional distribution of data points. FIG. 2A shows a number of data points, such as data point 202, distributed within the positive octant of a three-dimensional space defined by the three mutually orthogonal axes x 204, y 206, and z 208. In a three-dimensional distribution, each data point, such as data point 202, has an identity as well as values with respect to the x, y, and z axes. In order to construct a LOPS within a three-dimensional data-point distribution, the definition of a positive direction, or order, for data points is needed, as it was needed in the two-dimensional and one-dimensional cases. FIG. 2B illustrates a positive direction for construction of a LOPS in a three-dimensional distribution. In FIG. 2B, a data point 210 is shown positioned at the origin of three, mutually orthogonal, local axes xₗ, yₗ, and zₗ. Any data points within the positive octant bounded by the positive local axes xₗ, yₗ, and zₗ, is considered to be greater than, or of higher order than, data point 210, while data points within the negative octant bounded by the negative local axes x̅ₗ, y̅ₗ, z̅ₗ is considered to be less than, or of lower order than, data point 210, and data points in all six other octants are considered neither less than nor greater than data point 210. Thus, in FIG. 2B, data points 212 and 214, located within the positive octant, are considered to be greater than data point 210, and would be candidates for constructing the next edge of an order-preserving sequence passing through data point 210.

FIG. 2C shows two LOPS constructed from the three-dimensional distribution of data points shown in FIG. 2A. Projections of the two LOPS 216 and 218 onto the xy planes 220 and 222, respectively, are also shown in FIG. 2C. As the number of dimensions increases, the ratio of the length of one or more LOPS within a generalized distribution of data points to the number of data points within the distribution dramatically decreases. In a one-dimensional case, all the data points are members of a single LOPS within a data-point distribution. In the exemplary case of a two-dimensional distribution, a relatively large fraction of the data points of the distribution are included in one or more of the 40 different LOPS, but the length of a LOPS in a two-dimensional case is significantly less than the total number of data points within a two-dimensional distribution. In the three-dimensional case, illustrated in FIGS. 2A-C, the length of each LOPS is only three nodes, considerably less than the total number of data points shown in the distribution. Thus, as the number of dimensions increase, the constraints implied by a longest-order-preserving sequence become increasingly severe, and result in selecting a smaller and smaller percentage of the total number of data points within a distribution. Of course, in certain special distributions, the points may be arranged in any n-dimensional case so that all points occur within the LOPS, as in the one-dimensional case. However, for generalized distributions, the severity of the constraints increases with increasing dimensions. This is mathematically reasonable because, as can be seen by comparing FIG. 1B to FIG. 2B, the number of parameters necessary to determine the relative directions of one data point with respect to another increases linearly with an increase in dimensions. As the number of dimensions increases, and as the noise associated with each dimension increases, there are fewer and fewer points in each LOPS sequence, eventually leading to high-dimension cases in which the only LOPS sequences are trivial, single-point sequences. Such extrema represent over-constrained systems.

In this invention, LOPS are applied to DNA copy number data such as that obtainable from comparative genomic hybridization array data in which differentially labeled test and reference DNA samples are applied to an array of nucleic acid fragments corresponding to selected genes and selected tissue samples. From such data one can generate a DNA copy number matrix C=G×S that consists of the measured DNA copy numbers of n chromosomal loci or genes (G) over m samples (S). Under normal conditions, where no genomic aberrations exist in the test sample, DNA copy number data vectors should behave randomly, since any variations in the DNA copy number measurements would arise solely from experimental error.

For a continuous genomic segment G′ that is a subset of the set of all genes G (i.e., G′=(gᵢ . . . , gᵢ₊ₖ)⊂G) and a sample sⱼ element of samples S (sⱼεS), the vector vⱼ of DNA copy number measurements in segment G′ for sample sⱼ may be defined by

vⱼ=[C(i,j), . . . C(i+k,j)]

If no biological aberration has affected the continuous genomic segment G′, then the length of the LOPS within the set of vectors v₁, . . . , vₘ should not significantly deviate from the expected length of LOPS in a set of m random k-dimensional vectors. A significant deviation or variation of a LOPS may occur, for example, where the LOPS attains a value that would occur with a selected probability threshold, such as a probability of less than 10⁻⁴, less than 10⁻⁶, less than 10⁻¹⁰, less than 10⁻¹⁵, less than 10⁻²⁰ or (in certain embodiments) less than 10⁻³⁰, if m vectors of length k+1 were to be randomly drawn or selected. Thus, one would not expect to find specific LOPS that are significantly longer than the other LOPS within the vector set of continuous genomic segment G′ absent some chromosomal aberration. Therefore, any significantly long LOPS in this set of vectors indicates that some significantly consistent pattern exists in this submatrix of the DNA copy number matrix C.

Existence of a significantly long LOPS indicates that some specific order of the DNA copy numbers for a subset of the samples is persistent across several consecutive chromosomal loci. If the LOPS L is considered as a subset of samples S (i.e., L⊂S) that are incident to the identified LOPS L, a submatrix G′×L is defined. The submatrix G′×L may be called a genomic-continuous order-preserving submatrix or GC-OPSM of matrix C. Genomic continuity arises due to the sequential nature of the DNA copy number dataset for continuous genomic segment G′, and allows application of an efficient polynomial-time algorithm to identify all GC-OPSMs in a large dataset.

The biological interpretation of a GC-OPSM is that not only are most of the samples in LOPS L aberrant within the selected continuous genomic segment G′, but the penetrance of the aberration occurs at some gradual order within this subset of samples. In other words, if, for example, v₁<v₂< . . . vₗεL, then the aberration in continuous genomic segment G′ occurs with the lowest penetrance in sample sₗ and with the highest penetrance in sample sₗ (penetrance is the fraction of the cells in the samples that exhibit the chromosomal alteration).

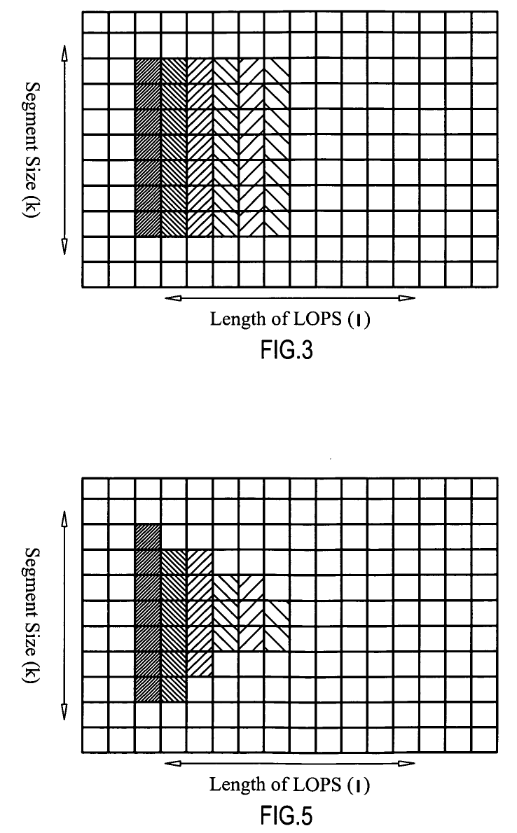

FIG. 3 provides a graphical illustration of the appearance of a LOPS in a DNA copy number data matrix. Segment size k is shown on the vertical axis and represents the size of continuous genomic segment G′, and l on the horizontal axis represents the length of LOPS L⊂S. Darker squares represent lower values, while lighter squares represent higher values.

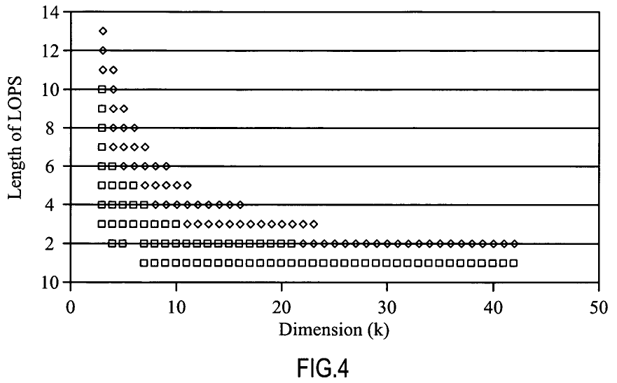

FIG. 4 illustrates use of the invention with a specific copy number data set reported by Pollack et al., “Genome-wide analysis of DNA copy number changes using cDNA microarrays”, Nature Genetics vol. 23(1), 41-46 (1999). This data set was generated from comparison of BT474 human breast cancer cells and normal female human leucocytes over 6,095 chromosomal loci across 41 different samples. FIG. 4 compares the occurrence of the lengths of LOPS within segments of k loci in the DNA copy number dataset of Pollack et al., with the expected occurrence of the lengths of LOPS in the same number (41) of random vectors of degree k, calculated by simulation over 10,000 random instances. LOPS was carried out using the algorithm discussed below. Diamond-shaped marks indicate LOPS found continuous genomic segments of the Pollack et al. copy number data set, while square-shaped marks show the length of LOPS found with simulated data using the same parameters. As can be seen from FIG. 2 4, there are numerous significantly long LOPS that appear in genomic segments of various lengths.

Application of LOPS approximations, such as α-LOPS and ε-distant LOPS can unveil even more genomic aberrations in DNA copy number data the appearance of which may be masked by experimental noise.

Genomic studies (Saito et al., “Evolution and stability of chromosomal DNA coamplified with the CAD gene”, Molecular and Cellular Biology, 9(6), 2445-2452 (1989)) suggest that tumor cells sometimes undergo a selection process in which the copy number of an amplified region (amplicon) typically increases in parallel with a decrease in the amplicon size. The framework of GC-OPSM in accordance with the invention may be extended to account for this biological model by allowing the size of the DNA copy numbers vectors in the LOPS to monotonically decrease, i.e. if v₁ [symbol] v₂ [symbol] . . . [symbol] v₁εL, then |v₁≧|v₂|≧ . . . ≧|v₁|).

FIG. 5 provides a graphical illustration of the appearance of a LOPS in a DNA copy number data matrix wherein DNA copy number vectors monotonically decrease as described above. Segment size k is shown on the vertical axis and represents the size of continuous genomic segment G′, and l on the horizontal axis represents the length of LOPS L⊂S. Darker squares represent lower values, while lighter squares represent higher values.

A specific embodiment of the invention uses the following algorithm for identifying LOPS:

n = |S|
For each segment G′ ⊂ G
l = FindLOPS(G′)
If Pval(G′,l,n) > threshold output G′

Function FindLOPS(G′)

l(1) = 1
n = |S|
for i = 2 to n

∘ lᵢ = max_{1≦j<i}(lⱼ:vⱼ < vᵢ) + 1

return maxᵢ(lᵢ)

Function Pval(G′,l,n)

m = |G′|
$return \frac{( \begin{matrix}n \\ l\end{matrix} )}{{( l ! )}^{( m - 1 )}}$

The function FindLOPS (G′) finds the longest order preserving sequence in the set of vectors defined by G′, the genomic segment. Various algorithms for identifying LOPS are known to those skilled in the art and may be used with the invention. One such algorithm, which extends easily to dimensions higher than two, is provided by Aldous and P. Diaconis in “Longest Increasing Subsequences: From Patience Sorting to the Baik-Dieft-Johansson Theorem”, Bull. Amer. Math. Soc. 36, 413-32

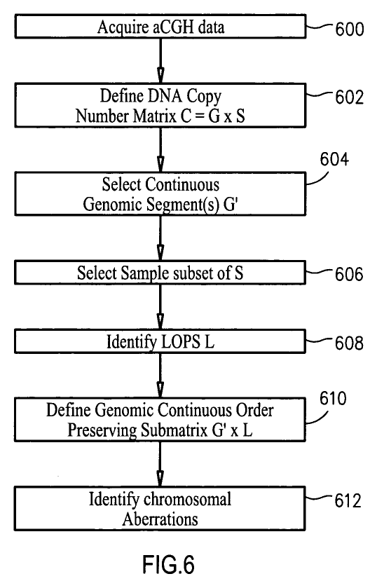

One embodiment of the subject methods is shown in the flow chart of FIG. 6. At event 600, comparative genomic hybridization array (aCGH) data is acquired or generated. This event involves designing and making a microarray suitable for generation of the aCGH data of interest, generating and labeling test and reference samples, hybridized to the microarray, and reading the array data. Alternatively, the array data may be provided from a stored location, such as computer storage, with the design and processing of the samples having already been previously carried out.

Many array platforms that may be used for generating aCGH data are well known in the art and may be used in event 600 (e.g., see Pinkel et al., Nat. Genet. (1998) 20:207-211; Hodgson et al., Nat. Genet. (2001) 29:459-464; and Wilhelm et al., Cancer Res. (2002) 62: 957-960). Such arrays may contain a plurality (i.e., at least about 100, at least about 500, at least about 1000, at least about 2000, at least about 5000, at least about 10,000, at least about 20,000, usually up to about 100,000 or more) of addressable features that are linked to a usually planar solid support. Features on a subject array usually contain a polynucleotide that hybridizes with, i.e., binds to, genomic sequences from a cell. CGH arrays typically have a plurality of different BACs, cDNAs, oligonucleotide primers, or inserts from phage or plasmids, etc., that are addressably arrayed on a substrate surface. CGH arrays thus typically contain surface bound polynucleotides that are about 10-200 bases in length, about 201-5000 bases in length, about 5001-50,000 bases in length, or about 50,001-200,000 bases in length, depending on the platform used and the nature of the CGH experiment. In particular embodiments, CGH arrays containing surface-bound oligonucleotide probes, i.e., oligonucleotides of 10 to 100 nucleotides and up to 200 nucleotides in length, may be useful with the invention.

The polynucleotides bound to the array will typically reflect a plurality of genes or chromosomal loci from a selected genome, as well as one or more tissue samples. Within the plurality of genes represented by the array is at least one continuous genomic segment as described above. The array may include polynucleotides representative of between 100 and 1000 genes, between 100 and 10,000 genes, or a larger number of genes depending upon the genome of interest. The array may include polynucleotides representative of between 1 and 10 different types of tissue sample, between 1 and 50 different types of tissue sample, between 1 and 100 different types of tissue sample, or a greater number of tissue samples.

Test and reference samples are prepared for use with the array of event 600 by obtaining and labeling test and reference genomic samples of nucleic acids. The test and reference samples may comprise, for example, the entire complement of chromosomes of a test cell and reference cell respectively (i.e., the chromosomes that make up the genome of a cell), fragmented versions thereof, amplified copies thereof, or amplified fragments thereof.

The test and reference cells used in event 600 may be from any two cells or sets of cells. In many embodiments, the test cell will have or be suspected of having a different phenotype compared to the reference cell. In a particular embodiment, test and reference cell pairs include cancerous cells, e.g., cells that exhibit increased genomic instability, and non-cancerous cells, respectively or cells obtained from a sample of tissue from a test subject, e.g., a subject suspected of having a chromosome copy number abnormality, and cells obtained from a normal, reference subject, respectively. Test and reference samples may be any cell of interest, including cells that contain or are suspected of containing an abnormal chromosome copy number.

The test and reference samples of nucleic acids used in event 600 may be labeled with the same label or different labels, depending on the actual assay protocol employed. For example, where each sample is to be contacted with different but identical arrays, the test and reference samples may be labeled with the same label. Alternatively, where both samples are simultaneously contacted with a single array, i.e., cohybridized, to the same array, solution-phase collections or populations of nucleic acids that are to be compared are generally distinguishably or differentially labeled with respect to each other.

The test and reference nucleic acid samples used in event 600 may be distinguishably labeled using various well known techniques, such as primer, extension, random-priming, nick translation, and the like. See, e.g., Ausubel, et al., Short Protocols in Molecular Biology, 3rd ed., Wiley & Sons 1995 and Sambrook et al., Molecular Cloning: A Laboratory Manual, Third Edition, 2001 Cold Spring Harbor, N.Y.). “Distinguishable” labels are labels that can be independently detected and measured, even when the labels are mixed. In other words, the amounts of label present for each of the labels are separately determinable, even when the labels are co-located on the same probe feature of an array surface. Suitable distinguishable fluorescent label pairs useful with the invention include Cy-3 and Cy-5 (Amersham Inc., Piscataway, N.J.), Quasar 570 and Quasar 670 (Biosearch Technology, Novato Calif.), Alexafluor555 and Alexafluor647 (Molecular Probes, Eugene, Oreg.), BODIPY V-1002 and BODIPY V1005 (Molecular Probes, Eugene, Oreg.), POPO-3 and TOTO-3 (Molecular Probes, Eugene, Oreg.), fluorescein and Texas red (Dupont, Boston Mass.) and POPRO3 TOPRO3 (Molecular Probes, Eugene, Oreg.).

In certain embodiments the test and reference nucleic acid composition may be of reduced complexity (such as about 20-fold less, about 25-fold less, about 50-fold less, about 75-fold less, about 90-fold less, or at about 95-fold less complex) in terms of total numbers of sequences present in the chromosome composition as compared to the entire chromosome complements of the test and references cells. Reduction in complexity can be achieved by using sequence specific primers in the generation of labeled nucleic acids, and by reducing the complexity of the chromosomal composition used to prepare the test and reference nucleic acid samples.

For hybridization in event 600, the test and reference nucleic acid samples are contacted to an array surface under conditions such that nucleic acid hybridization to the surface-bound probes can occur. The test and reference samples may be applied in a suitable buffer containing 50% formamide, 5×SSC and 1% SDS at 42° C., or in a buffer containing 5×SSC and 1% SDS at 65° C., both with a wash of 0.2×SSC and 0.1% SDS at 65° C. In many embodiments the test and reference nucleic acids may be contacted with an array surface simultaneously.

Standard hybridization techniques may be used in event 600, which may vary in stringency as desired. In certain embodiments, highly stringent hybridization conditions may be employed as described above. Kallioniemi et al., Science 258:818-821 (1992) and WO 93/18186 describe conventional CGH techniques. Several guides to general techniques are available, e.g., Tijssen, Hybridization with Nucleic Acid Probes, Parts I and II (Elsevier, Amsterdam 1993). For a descriptions of techniques suitable for in situ hybridizations, see Gall et al. Meth. Enzymol., 21:470-480 (1981) and Angerer et al. in Genetic Engineering: Principles and Methods Setlow and Hollaender, Eds. Vol 7, pgs 43-65 (plenum Press, New York 1985).

Array data may measured or determined in event 600 by standard detection techniques may be used in reading the hybridization data from an array surface. Where fluorescent labeling of the test and reference nucleic acids is used, reading of the hybridized array may be achieved by illuminating the array and reading the location and intensity of resulting fluorescence at each feature of the array to detect binding complexes on the array surface. A scanner, such as the AGILENT MICROARRAY SCANNER available from Agilent Technologies, Palo Alto, Calif., may be used for measuring data. Arrays may be read by other methods such as other optical techniques (for example, detecting chemiluminescent or electroluminescent labels) or electrical techniques (where each array feature is provided with an electrode to detect hybridization). In the case of indirect labeling, subsequent treatment of the array with the appropriate reagents may be employed to enable reading of the array. Some methods of detection, such as surface plasmon resonance, do not require any labeling of the probe nucleic acids, and are suitable for some embodiments.

At event 602, a DNA copy number matrix is defined or otherwise created from the aCGH data generated in event 600. A DNA copy number matrix C=G×S consists of the measured DNA copy numbers of n chromosomal loci or genes G over m samples S. The matrix C contains one column for every sample in S and one row for every genomic locus. The i,jth entry C(i,j) represents the measurement at locus i for sample j.

At event 604, a continuous genomic segment G′ is selected. Continuous genomic segment G′ is a subset of genes G of step 602, such that G=(gᵢ . . . , gᵢ₊ₖ)⊂G.

At event 606 a sample element or subset of samples S=s1, s2, s3 . . . (sⱼεS) is selected.

At event 608, a LOPS L is identified from the submatrix defined by continuous genomic segment G′ and sample element sⱼ from events 604 and 606 respectively. This event may be carried out using a variety of algorithms including the methods described in Aldous and Diaconis.

At event 608, a LOPS L is identified from the submatrix defined by continuous genomic segment G′ and sample element sⱼ from events 604 and 606 respectively. This event may be carried out using a variety of algorithms including the methods described in Aldous and Diaconis.

At event 610, a genomic continuous order preserving submatrix G′×L is defined or otherwise created. L is a subset of samples that contains only the indices of the LOPS found over G′.

At event 612, identification of chromosomal aberration is carried out. As noted above, if no biological aberration has affected the continuous genomic segment G′, then the length of the LOPS within the set of vectors v₁, . . . , vₘ should not significantly deviate from the expected length of LOPS in a set of m random k-dimensional vectors. Thus, in event 612 the LOPS of events 608-610 (generated from G′×sⱼ submatrix) are compared to LOPS generated from random vectors of degree k over the same sample sⱼ. The presence of any LOPS from the continuous genomic segment G′ that is significantly longer than the LOPS from the random vectors indicates a chromosomal aberration. A significantly longer LOPS can be determined by randomly drawing m vectors of length k+1, as noted above, with the LOPS being significant if the attained p-value is smaller than a user-defined or selected probability threshold.

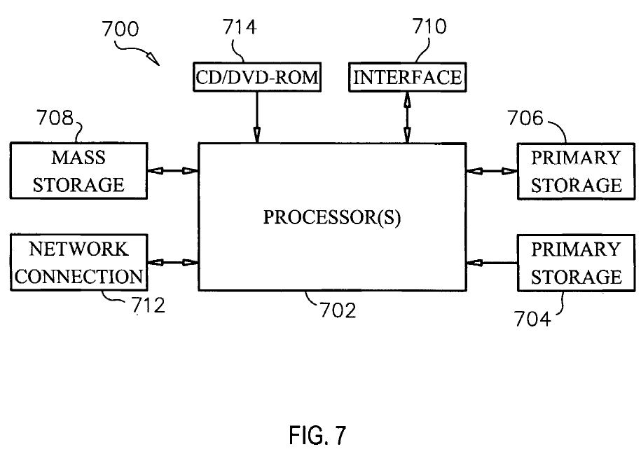

The identification of chromosomal aberrations from aCGH data using LOPS in accordance with the invention may be carried out with a computer system such as system 700 shown in FIG. 7. The computer system 700 includes any number of processors 702 (also referred to as central processing units, or CPUs) that are coupled to storage devices including primary storage 704 (typically a random access memory, or RAM), primary storage 706 (typically a read only memory, or ROM). As is well known in the art, primary storage 704 acts to transfer data and instructions uni-directionally to the CPU, and primary storage 706 is used typically to transfer data and instructions in a bi-directional manner. Both of these primary storage devices may include any suitable computer-readable media containing program elements capable of performing the data smoothing operations described above.

A mass storage device 708 is also coupled bi-directionally to CPU 702 and provides additional data storage capacity and may include any of the computer-readable media described above. Mass storage device 708 may be used to store programs, data and the like and is typically a secondary storage medium such as a hard disk that is slower than primary storage. It will be appreciated that the information retained within the mass storage device 708, may, in appropriate cases, be incorporated in standard fashion as part of primary storage 706 as virtual memory. A specific mass storage device such as a CD-ROM 714 may also pass data uni-directionally to the CPU 702.

CPU 702 is also coupled to an interface 710 that includes one or more input/output devices such as such as video monitors, track balls, mice, keyboards, microphones, touch-sensitive displays, transducer card readers, magnetic or paper tape readers, tablets, styluses, voice or handwriting recognizers, or other well-known input devices such as, of course, other computers. Finally, CPU 702 optionally may be coupled to a computer or telecommunications network using a network connection as shown generally at 712. With such a network connection, it is contemplated that the CPU 702 might receive information from the network, or might output information to the network in the course of performing the above-described method steps. The above-described devices and materials will be familiar to those of skill in the computer hardware and software arts.

The hardware elements described above may implement the instructions of multiple software modules for performing the data smoothing operations of this invention. For example, instructions for computing long order preserving subsets in set of vectors and/or for generating graphical representations of long order preserving subsets of data, may be stored on mass storage device 708 or 714 and executed on CPU 702 in conjunction with primary memory 706.

In addition, embodiments of the present invention further relate to computer readable media or computer program products that include program instructions and/or data (including data structures) for performing various computer-implemented operations. The media and program instructions may be those specially designed and constructed for the purposes of the present invention, or they may be of the kind well known and available to those having skill in the computer software arts. Examples of computer-readable media include, but are not limited to, magnetic media such as hard disks, floppy disks, and magnetic tape; optical media such as CD-ROM, CDRW, DVD-ROM, or DVD-RW disks; magneto-optical media such as “floptical” disks; and hardware devices that are specially configured to store and perform program instructions, such as read-only memory devices (ROM) and random access memory (RAM). Examples of program instructions include both machine code, such as produced by a compiler, and files containing higher level code that may be executed by the computer using an interpreter.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

What is claimed is:

1. A method for tumor progression analysis in comparative genomic hybridization data, the method comprising:
acquiring comparative genomic hybridization data;
identifying a long order preserving subset within the comparative genomic hybridization data; and
identifying a chromosomal aberration associated with the long order preserving subset.

2. The method of claim 1, wherein the comparative genomic hybridization data is comparative genomic hybridization array data.

3. The method of claim 2, further comprising:
determining a nucleic acid copy number matrix from the comparative genomic hybridization array data; and
identifying the long order preserving subset from nucleic acid copy number data vectors of the nucleic acid copy number matrix.

4. The method of claim 3, further comprising:
selecting a continuous genomic segment and samples representing a subset of the nucleic acid copy number matrix; and
identifying the long order preserving subset from nucleic copy number data vectors associated with the selected genomic segment and samples.

5. The method of claim 4, further comprising identifying a genomic continuous order preserving submatrix associated with the selected genomic segment and samples.

6. A method for tumor progression analysis in comparative genomic hybridization array data, the method comprising:
acquiring comparative genomic hybridization array data;
defining a DNA copy number matrix C=G×S for the comparative genomic hybridization array data, wherein the matrix C comprises measured DNA copy number vectors for n chromosomal loci G over m samples S;
identifying a long order preserving subset within the measured DNA copy number vectors; and
identifying a chromosomal aberration associated with the long order preserving subset.

7. The method of claim 6, further comprising:
selecting a continuous genomic segment G′ that is a subset of chromosomal locsi G;
selecting a sample set sⱼ that is an element of samples S, such that continuous genomic segment G′ and sample set sⱼ define a submatrix of DNA copy number matrix C; and
identifying the long order preserving subset from the DNA copy number vectors of the submatrix defined by the continuous genomic segment G and sample set sⱼ.

8. The method of claim 7, further comprising defining a genomic continuous order preserving submatrix G′×L of DNA copy number matrix C, wherein L is the long order preserving subset.

9. The method of claim 8, further comprising determining the penetrance of the chromosomal aberration within the subset of samples L.

10. A method for tumor progression analysis in comparative genomic hybridization array data, the method comprising:
acquiring comparative genomic hybridization array data;
defining a DNA copy number matrix C=G×S for the comparative genomic hybridization array data, wherein the matrix C comprises measured DNA copy number vectors for n chromosomal loci G over m samples S;
selecting a continuous genomic segment G′=(gᵢ, . . . gᵢ₊ₖ) that is a subset of chromosomal locsi G, wherein gᵢ . . . gᵢ₊ₖ represent individual chromosomal loci;
selecting a sample set sⱼ that is an element of samples S, wherein vector vⱼ for a DNA copy number measurement in continuous genomic segment G′ for the sample sⱼ is represented by vᵢ=[C(i,j), . . . C(i+k, j)]; and
identifying the long order preserving subset from a set of vectors v₁, . . . vₘ for the genomic segment G′.

11. The method of claim 10, further comprising defining a genomic continuous order preserving submatrix G′×L wherein L is the long order preserving subset of samples S and G′ is the selected continuous genomic segment.

12. A tumor progression analysis system for comparative genomic hybridization data, the system comprising:
means for inputting comparative genomic hybridization data;
means identifying a long order preserving subset within the comparative genomic hybridization data; and
means for identifying a chromosomal aberration associated with the long order preserving subset.

13. The system of claim 12, wherein the comparative genomic hybridization data is comparative genomic hybridization array data.

14. The system of claim 13, further comprising:
means for determining a nucleic acid copy number matrix from the comparative genomic hybridization array data; and
means for identifying the long order preserving subset from nucleic acid copy number data vectors of the nucleic acid copy number matrix.

15. The system of claim 14, further comprising:
means for selecting a continuous genomic segment and samples representing a subset of the nucleic acid copy number matrix; and
means for identifying the long order preserving subset from nucleic copy number data vectors associated with the selected genomic segment and samples.

16. The system of claim 15, further comprising means for identifying a genomic continuous order preserving submatrix associated with the selected genomic segment and samples.
